(19) United States
(12) Patent Application Publication
(10) Pub. No.: US 2012/0132218 A1
(43) Pub. Date: May 31, 2012

(54) SMOKELESS TOBACCO CESSATION PRODUCT

(76) Inventors: Lee B. Lewis, Fernandina Beach, FL (US); Meredith L. Lewis, Fernandina Beach, FL (US)

(21) Appl. No.: 12/955,372

(22) Filed: Nov. 29, 2010

(51) Int. Cl. A61K 9/14; A61K 31/4188

(52) U.S. Cl. 131/270

(57) ABSTRACT

A product useful in assisting one to stop using smokeless tobacco products includes small sponge-like particles having a size similar to shredded tobacco, and moisture content similar to shredded tobacco. The particles further include a flavoring agent and, preferably, a pharmaceutically acceptable stimulant. The particles, preferably shredded sea sponge, can be placed in the mouth as a substitute for shredded tobacco products. They have the same feel as shredded tobacco products, provide a stimulate effect similar to a tobacco product, but do not have any adverse health effects.

### BACKGROUND OF THE INVENTION

Like cigarettes, smokeless tobacco is both addictive and harmful. The product is associated with various types of cancer and other oral diseases, but, because of the addictive nature it is difficult for one to stop using smokeless tobacco.

Smokeless tobacco has both a chemical and a physical appeal. Users become accustomed to having the product in their mouths.

There are a variety of different items that use nicotine as a way to assist in reducing the urge to use tobacco products. However, the nicotine per se is harmful. Therefore, it is preferable to use a smoke cessation product that does not rely on nicotine.

### SUMMARY OF THE INVENTION

The present invention is premised on the realization that one can reduce the urge to use smokeless tobacco products by substituting a soft sponge-like material coated with a flavored substance for the tobacco product. The sponge-like product preferably is maintained moist like shredded tobacco, formed in small particles like shredded tobacco, and, soft like shredded tobacco. The product, when placed in the mouth feels like tobacco and has the same physical appeal as smokeless tobacco.

More preferably, the product of the present invention further may include a chemical stimulant that is pharmaceutically acceptable, and, in particular, caffeine. The product can further include other components such as preservatives, sweeteners, and cooling agents, to simulate smokeless tobacco. In particular, the flavoring component can be a wide variety of different flavorings, such as menthol similar to menthol flavored smoking tobacco, a tobacco flavoring, other flavorings such as coffee, and the like.

The objects and advantages of the present invention will be further appreciated in light of the following detailed description and drawings in which:

### BRIEF DESCRIPTION OF THE DRAWING

FIG. 1 is a cross-sectional view of a container holding the product of the present invention;

FIG. 2 is an enlargement of a piece of a sponge particle.

### DETAILED DESCRIPTION

The present invention includes a soft particulate material which is intended to have the feel of long cut, shredded tobacco. The individual particles 12 have a maximum dimension 14 less than about 1 inch, and preferably having a maximum dimension 14 of about ¾ of an inch down to ⅛ of an inch, and a smallest dimension 15 of about ⅛ of an inch to ¾ of an inch. The particle size, of course, will vary widely. Some particles will fall below the range and a few particles may be larger than this. But, on average the maximum size of the particles should be less than 1 inch and preferably about ¾ of an inch to ⅛ of an inch.

The particles themselves should be soft and sponge-like, much like shredded tobacco. The particles can be formed from a wide variety of different products. However, sponge is ideal for use in the present invention, and, in particular sterilized sea sponge is ideal for use in the present invention.

The sea sponge particles are formed by shredding with, for example, a rotary blade granulating machine. One such machine uses a five-knife stainless steel granulating apparatus. However, any apparatus that will affectively cut the sponge to the appropriate size can be used for the present invention. The size is intended to be similar to long cut tobacco products.

Since the preferred material is a natural product, the sponge needs to be sterilized and is preferably sterilized using, for example, 70-95% lab grade ethanol to kill any bacteria or other microbes on the sponge. This is then press dried and air dried to remove the alcohol.

The particulate material is coated with an aqueous flavoring composition or mixture. A wide variety of different flavoring agents can be used, in particular, flavoring agents that simulate smokeless tobacco products. Since many smokeless tobacco products are flavored, these same flavorings, natural and artificial, can be used. In particular, wintergreen flavor is typically used on smokeless tobacco products and, accordingly, is particularly suited for use with the present invention. In addition, other flavoring agents include natural tobacco flavoring, various types of mints, and less traditional flavorings such as coffee. Sweeteners, either natural sugar or artificial, such as sucralose, can also be added to enhance the taste.

The flavoring composition can include additional additives. The product can include a pharmaceutically acceptable stimulant, which will increase the appeal of the product. In particular, caffeine can be added to the present invention in an amount that is pharmaceutically safe and acceptable, and in an amount that will not irritate the mucous lining of the mouth.

Further, the present invention can include vitamins, such as vitamin B12, vitamin C, niacin, beta alanine, and the like, as well as preservatives such as sodium citrate and potassium sorbate. Thickening agents such as xanthan gum can be used to improve the mouth feel of the product. This will provide sublingual delivery of the vitamins and/or stimulant.

The product is formulated by forming and sterilizing the sponge particles and combining these with an aqueous solution of the flavorings and other additives and tumbling these for a period of time, about 10 minutes. A vacuum can be then pulled inside the tumbler to remove most, if not all, of the air. Once completed the machine tumbles for another period of time, such as 10 minutes. The product is then removed from the tumbler and placed in containers for consumer use.

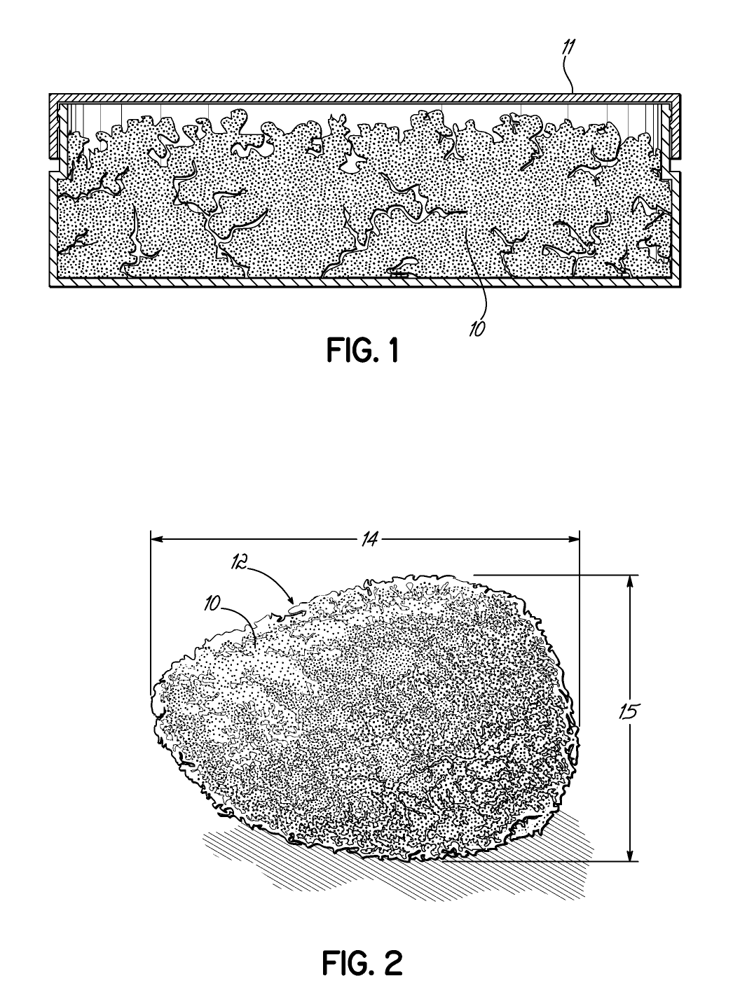

It is important that the product maintain its moisture content so, when used, it has the feel of shredded tobacco. Accordingly, as shown in FIG. 1, the shredded coated sea sponge 10 is packaged in an air tight container 11, which maintains the aqueous coating composition in the sponge. It should be noted that the amount of the aqueous coating composition maintains the particles of sponge moist, but does not leak out and leave free liquid within the container. This amount can, of course, vary depending upon the particular sponge that is employed.

The product is used in the same manner as shredded tobacco products. An amount of the product, generally 1 to 2 grams, is simply placed in the mouth, the same location one places shredded tobacco. This will provide the same feel as shredded tobacco. The wintergreen flavoring, along with the stimulant and any other additives such as sweeteners and vitamins, will slowly leach out of the sponge, reducing one's urge to use a tobacco product, and, at the same time without causing any adverse effects. Eventually, the addiction to the tobacco should decrease to the point where the product is unnecessary. However, the product can simply be used as a source of stimulant. Since it has no adverse side effects, it may be employed by individuals who simply desire the caffeine and do not use smokeless tobacco products.

This has been a description of the present invention along with the preferred method of practicing the present invention. However, the invention itself should only be defined by the appended claims, WHEREIN WE CLAIM:

What is claimed is:

1. A method of reducing the urge to use tobacco products comprising placing in the mouth
a plurality of particles said particles having a soft sponge-like feel and a moist feel; and
a flavoring agent.

2. The method claimed in claim 1 wherein said particles comprise shredded sponge.

3. The method claimed in claim 2 wherein said shredded sponge comprises shredded sea sponge.

4. The method claimed in claim 3 wherein said particles further include a pharmaceutically accepted stimulant.

5. The method claimed in claim 4 wherein said stimulant comprises caffeine.

6. The method claimed in claim 5 wherein said particles are stored in an air tight container prior to use.

7. The method claimed in claim 1 wherein said particles have an average maximum size less than 1 inch.

8. The method claimed in claim 6 wherein said particles further include a sweetener.

9. The method claimed in claim 8 wherein said particles further include a vitamin.

10. The method claimed in claim 9 wherein said vitamin comprises vitamin B12.

11. A smoke cessation product comprising sponge particles having an average maximum size of less than 1 inch moistened with an aqueous solution including a pharmaceutically acceptable stimulant and a flavoring agent.

12. The composition claimed in claim 1 wherein said composition is maintained in an air tight container.
